(19) United States
(12) Patent Application Publication
(10) Pub. No.: US 2025/0152187 A1
(43) Pub. Date: May 15, 2025

(54) METHOD OF MANUFACTURING A CUTTING HEAD, A CUTTING HEAD, AND A PERFORATOR

(71) Applicant: adeor Medical AG, Valley (DE)

(72) Inventors: Johann Fersterer, Valley (DE); Fabio Von Zeppelin, Valley (DE)

(73) Assignee: adeor Medical AG, Valley (DE)

(21) Appl. No.: 18/839,965

(22) PCT Filed: Feb. 24, 2023
(86) PCT No.: PCT/EP2023/054716

§ 371 Date: Aug. 20, 2024

(30) Foreign Application Priority Data
Feb. 24, 2022 (GB) 2202578.7

(51) Int. Cl. A61B 17/16; A61B 17/00

(52) U.S. Cl. CPC A61B 17/1695; A61B 17/1615; A61B 2017/00526

(57) ABSTRACT

A method of manufacturing a cutting head for a perforator for cutting bone tissue is provided. The method comprising the steps of: machining a cutting head comprising a plurality of cutting edges from a material having a Rockwell C hardness HRC of less than 30 HRC; and surface finishing the machined cutting head, in which the method does not comprise hardening. A cutting head is also provided.

The present disclosure relates to a method of manufacturing a cutting head for a perforator, to a cutting head, and to a perforator. In a particularly preferred embodiment, the disclosure relates to a method of manufacturing a cutting head for a perforator for cutting a hole in a skull of a human or animal.

### BACKGROUND

In order to carry out surgical operations inside the cranial cavity, it is necessary to first obtain access to the cranial cavity by drilling one or more holes through the bone of the skull (the cranium). The process of drilling a hole through the skull is called trepanation. Trepanation is a difficult and delicate procedure, as sufficient force must be applied to the drill head for it to advance through the hard layers of bone tissue, but the drill head must be halted immediately after perforation of the skull in order to avoid the drill head damaging the dura mater or soft tissue inside the cranium.

Prior art perforators have typically been similar to conventional drills, in that a forward force or pressure must be applied to the drill to drive the drill head through the skull. A major risk with such prior art designs is that when the cranium is perforated, the forward force or pressure on the drill head can drive the drill head inwards through the newly created hole and into the cranium, potentially causing potential damage to the dura mater.

Attempts to reduce this risk of damage to the dura mater have focused on stopping rotation of the drill head as soon as the cranium is perforated, so that the rotating cutting or drill head does not tear or otherwise damage the dura mater. In order to provide a perforator in which the rotation of the drill head stops as soon as the cranium has been perforated, cranial perforators are provided in the prior art in which the drill head is not permanently coupled to a drive shaft of an electric drill. However, these prior art cutting heads do not mitigate the risk of damage to the dura mater if the drill head does not disconnect from the drive shaft in time.

In addition, producing the cutting heads of prior art perforators is time-consuming and labour-intensive. In particular, the cutting heads are typically produced by: drilling and milling to produce the correct shape; hardening to ensure that the drill head is sufficiently hard to drill bone tissue; grinding to sharpen the cutting edges; deburring; and finally surface finishing.

The inventors have appreciated the need for a simpler method of manufacturing a cutting head for a perforator for cutting bone tissue. The inventors have also appreciated the need for a perforator with a cutting head that is easily manufacturable, while functioning well and providing significant functional reserves.

### SUMMARY OF INVENTION

The disclosure provides a method of manufacturing a cutting head for a perforator, a cutting head, and a perforator, as defined in the appended independent claims, to which reference should now be made. Preferred or advantageous features of the disclosure are set out in dependent subclaims.

In a first aspect, the disclosure provides a method of manufacturing a cutting head for a perforator, the method consisting of: machining a cutting head comprising a plurality of cutting edges from a material having a Rockwell C hardness HRC of less than 30 HRC; and surface treating the machined cutting head.

Unlike in known methods of manufacturing cutting heads, in the present method, the cutting head may be machined directly from a starting material to a cutting head having substantially the final shape desired for the cutting head. This is because the cutting head is machined from a material having a relatively low HRC of less than 30 HRC.

The conventional wisdom in the art is that in order to be suitable for cutting bone, perforator cutting heads must be hardened to over 50 HRC and then sharpened by grinding. The inventors have surprisingly found, however, that a cutting head machined from a material with a hardness of less than 30 HRC, once machined and without being hardened, still cuts bone tissue well, and functions as well as, or better than, a conventional cutting head formed from harder material.

According to the method of the present disclosure, after the cutting head has been machined from the starting material, it is only surface treated to produce the final cutting head. In this way, time-intensive and costly secondary treatment of the machined cutting head is avoided. There is no need for grinding, or other manual processes, once the starting material has been machined into shape.

Thus the present method comprises only the steps of machining the starting material into the shape of the cutting head, and then carrying out a surface treatment step on the machined cutting head. The method does not comprise a hardening or tempering step. The method also does not comprise separate steps of grinding, or deburring.

The material having a Rockwell C hardness HRC of less than 30 HRC is preferably provided as a blank having a size suitable for machining into a cutting head for a perforator. The material having a Rockwell C hardness HRC of less than 30 HRC may be referred to as a blank, or a starting material, or a blank of starting material.

Perforator cutting heads in the prior art are typically formed from metal having, after hardening, a Rockwell C hardness of at least 50 HRC, for example 55+/−3 HRC for conventional perforator cutting heads made from 1.4034 stainless steel. The reason for this is a long-held prejudice in the art that the perforator cutting edges require a high hardness in order to cleanly cut through bone, when a perforator is to be used for processes such as trepanation. In order to achieve this high hardness cutting head, prior art methods have typically used a high-hardness starting material and included several energy—and labour-intensive processing steps, including hardening or tempering. In the present invention, however, the inventors have provided a perforator cutting head which performs well even when manufactured from a starting material with a hardness of less than 30 HRC, using a significantly simplified manufacturing process, without a hardening or tempering step.

In known methods of manufacturing cutting heads, cutting heads need to be hardened, which slows down the manufacture of the cutting heads, and increases manufacturing costs. Further, because of the hardening step, separate grinding or sharpening steps are also required before prior art cutting heads are ready for use.

Manual processes, including grinding, often lead to inconsistencies in the final products. By removing the need for any manual processes and/or grinding, the present method may lead to more consistent cutting heads, and which function in a more reproducible manner.

An example known method of manufacturing a cutting head for a perforator includes the following steps: turning and milling from a blank; hardening and tempering; second machining such as grinding; deburring (manually and/or by machine); and surface finishing. Between the machining steps there are additional control and cleaning steps. It is clear that the method according to the present disclosure is much simpler and quicker when compared to this exemplary known method, eliminating the need for many of the previously required steps.

It is noted that “surface treating”, or “surface finishing”, according to the present disclosure refers only to surface treatments, i.e. steps which only superficially modify the machined cutting head. Therefore, “surface treating” steps are finishing and/or polishing steps which relate to surface finishing processes which improve the surface finish of the machined cutting head without re-shaping the workpiece or substantially affecting its hardness. The step of surface treating the machined cutting head may be a step of finishing and/or polishing the machined cutting head.

The hardness of the machined cutting head will be substantially the same as the hardness of the starting material, as there is no hardening step between the milling of the starting material and the finished cutting head. In the prior art, tension or warping is usually introduced into the material of cutting heads by heat treatment (e.g. hardening). As no heat treatment is conducted in the method according to the present disclosure, tension or warping of the material is prevented.

Hardness according to the disclosure is measured in HRC, Rockwell hardness C, which is commonly used to scale the hardness of metals and metal alloys, in particular steel. However, any other suitable hardness scale which allows for conversion into HRC may be employed by the person skilled in the art—for example, Rockwell hardness B, Brinell hardness, or Vickers hardness.

HRC may be measured according to DIN EN ISO 14577-1:2015-11.

In general, Rockwell hardness is calculated according to the following equation: HR=N−hd, in which HR is an arbitrary, dimensionless number (the Rockwell hardness), N and h are scale factors depending on which Rockwell scale is being used, and d is the indentation depth in mm.

To determine the indentation depth, Rockwell hardness testing comprises four steps: loading with an initial force of 98 N; loading with a main load; applying the main load for a dwell time sufficient for indentation to terminate; and releasing the load. The initial and main loads are applied by an indenter—in HRC testing, the indenter is a spheroconical diamond called a Brale indenter, N is 100, h is 500, and the main load is 150 kgf (1471 N). The Brale indenter is a conical diamond of 120°+0.35° included angle and a tip radius of 0.20±0.01 mm.

The cutting head may be machined from the starting material by drilling and/or milling. Optionally, the machining is by computer numerical control CNC drilling and milling. Advantageously, by using CNC machining, high consistency may be achieved by reducing the risk of manual working introducing inconsistencies.

The step of machining the cutting head from the starting material preferably comprises one or more mechanical machining processes. The step of machining may comprise or consist of one or more of drilling, milling, single-point cutting or multi-point cutting. The step of machining does not involve grinding.

Grinding is an important step in the manufacture of prior art perforators. Prior art perforators are ground in a relatively complex way in special CNC grinding machines, which are capable only of grinding. Hardened raw parts, i.e. raw parts which have been machined and hardened, are clamped in jigs and then ground automatically. In contrast, in the method of the present disclosure, no grinding is required, the cutting head is only drilled and milled-grinding is not considered “machining” in the sense of the present disclosure.

Optionally, the cutting head and the cutting edges are machined to their final shape in a single machining step, using a single setup. In other words, a blank of starting material is set up for machining, and the blank is then machined into a cutting head without removing the workpiece (the blank being machined) from the machining apparatus. This is in contrast to prior art methods in which workpieces must be removed from the machining apparatus for hardening and tempering before being set up again for further processing steps. Advantageously, by using a single setup for machining, time is saved and reproducibility is further improved.

Optionally, the step of surface treating the machined cutting head comprises finishing or polishing. Optionally, the step of surface treating the machined cutting head comprises one of: vibratory finishing; and electrolytic polishing. The step of surface treating the machined cutting head may comprise either vibratory finishing or electrolytic polishing. Vibratory finishing is an automated process for removing burrs, rounding edges, homogenising the surface, and creating a high-quality optical appearance. Vibratory finishing allows for the cutting head to slide better against other components of the perforator.

The rounding of edges which takes place during vibratory finishing and electrolytic polishing affects the sharpness of the cutting edges. Thus, by controlling the vibratory finishing and/or electrolytic polishing steps, the sharpness of the cutting edges may be adjusted to a desired sharpness. By carrying out vibratory finishing and/or electrolytic polishing, the surface finish of the machined cutting head may be improved, so that after surface treating the machined cutting head, the cutting head is ready for use in a perforator without any further processing.

Alternatively, the step of surface treating the machined cutting head may comprise, or consist of, blasting with dry ice, blasting with glass beads or other blasting material, or brushing directly in a CNC machine. Brushing directly in the CNC machine may be achieved by rotating brushes made of plastic or metal against the cutting head. This has the advantage that the resulting cutting edges are finished in the CNC machine, avoiding the need to move the machined cutting head into a vibratory finishing device or an electrolytic polishing bath.

The following are not considered to fall under the definition of surface treating the machined cutting head: turning; milling; grinding; hardening and tempering; and passivation. Thus the surface treating step does not comprise any of: turning; milling; grinding; and hardening and tempering.

Preferably the surface treatment step does not comprise passivation.

Optionally, the starting material from which the cutting head is machined has a HRC of less than 25 HRC, optionally less than 20 HRC. Further optionally, the starting material has a HRC of about 18 HRC, for example an HRC of between 16 and 20 HRC, or between 17 and 19 HRC, or between 17.5 and 18.5 HRC. The lower the HRC hardness, the easier it is to machine the cutting head in a single step. However, this must be balanced with the requirement for sufficient cutting resilience when using the perforator cutting head to cut through bone tissue.

It is noted that a hardness of the machined cutting head may be substantially the same as the hardness of the starting material, as there is no hardening or tempering.

The inventors have surprisingly found that even with a HRC of about 18 HRC, the cutting head has sufficient cutting resilience to be suitable for trepanation, in particular for single use trepanation which is common for perforators.

Optionally, the material is a stainless steel, in particular an austenitic stainless steel. Austenitic stainless steel may be a particularly suitable material for use in the method according to the present disclosure because austenitic steel cannot be hardened via heat treatment. Austenitic steel has therefore not been used in perforators. However, it has been found to be suitable for use in the method of the present disclosure, which does not comprise a hardening step (and in particular, a step of hardening by heat treatment). The corrosion resistance of austenitic steels is beneficial when used in perforators.

While the methods of the present disclosure may be carried out with any suitable material, particularly any stainless steel, given that there is no hardening step, austenitic steel which is not hardenable (by heat treatment) and corrosion resistant may be particularly suitable.

Further optionally, the stainless steel is one of: 1.4301; 1.4305; 1.4306; 1.4307; 1.4310; 1.4401; 1.4404; 1.4435; and 1.4441. In preferred embodiments, the stainless steel is 1.4404. Advantageously, stainless steel is readily available and relatively inexpensive, but its properties are suitable for surgical uses. Although 1.4404 is more expensive than some other stainless steels, such as 1.4034, by dispensing with the need for unnecessary process steps, the production makes the cutting heads relatively inexpensive.

In the present disclosure, the nomenclature for different stainless steel compositions is according to DIN steel standards. Alternatively, the material may be titanium, or a titanium alloy.

Optionally, machining the cutting head may comprise machining a connector portion. The connector portion may be at least one slot and/or at least one projection. The connector portion may be machined in the same setup, and using the same mechanical machining process(es), as the rest of the cutting head, e.g. by drilling, milling, single-point cutting or multi-point cutting.

In a second aspect, the disclosure provides a method of manufacturing a cutting head for a perforator, the method comprising the steps of: machining a cutting head comprising a plurality of cutting edges from a material having a Rockwell C hardness HRC of less than 30 HRC; and surface finishing the machined cutting head, in which the method does not comprise hardening by heat-treatment.

As set out above, perforator cutting heads are typically produced by: drilling and milling to produce the correct shape; hardening (by heat treatment) to ensure that the drill head is sufficiently hard to drill bone tissue; grinding to sharpen the cutting edges; deburring; and finally surface finishing.

Although the method refers to there being no hardening by heat treatment, it is noted that preferably, the method comprises no hardening step at all. Hardening by heat treatment is a standard step, which typically follows machining (e.g. drilling and milling) the cutting head, and may be dispensed with in the method of the present disclosure. In other words, the method does not comprise a step of hardening by heat treatment, and may not comprise any step of hardening.

As such, the disclosure may provide a method of manufacturing a cutting head for a perforator, the method comprising the steps of: machining a cutting head comprising a plurality of cutting edges from a material having a Rockwell C hardness HRC of less than 30 HRC; and surface finishing the machined cutting head, in which the method does not comprise a step of hardening.

The method may further not comprise a step of grinding the machined (and unhardened) cutting head. As set out above, grinding a hardened cutting head is a standard step in the prior art processes for making cutting heads for perforators, typically following, and necessitated by, heat treatment. This step may be dispensed with in the method of the present disclosure (as there is no heat treatment step).

The material may be a metal. The material may be a stainless steel. In particular, the material may be an austenitic stainless steel. As set out above, austenitic stainless steel may be a particularly suitable material for use in the method according to the present disclosure because austenitic steel cannot be hardened via heat treatment but is corrosion resistant.

The stainless steel may be one of 1.4301; 1.4305; 1.4306; 1.4307; 1.4310; 1.4401; 1.4404; 1.4435; and 1.4441.

The method preferably comprises only the steps of: machining the cutting head from a material having a Rockwell C hardness HRC of less than 30 HRC, and surface finishing the machined cutting head.

The material may have a HRC of less than 25 HRC, or of less than 20 HRC, or of between 16 and 20 HRC, or between 17 and 19 HRC, or between 17.5 and 18.5 HRC. In one particular example, the material may have a HRC of about 18 HRC.

The step of machining the cutting head from the starting material (the material having a Rockwell C hardness HRC of less than 30 HRC) preferably comprises one or more mechanical machining processes. The step of machining may comprise or consist of one or more of drilling, milling, single-point cutting or multi-point cutting.

The cutting head is preferably machined from a blank of the starting material by drilling and/or milling. Optionally, the machining is by computer numerical control CNC drilling and milling.

The cutting head and the cutting edges may be machined to their final shape in a single machining step, using a single setup.

The step of surface finishing the machined cutting head may comprise finishing or polishing the cutting head and/or the cutting edges. The step of surface finishing the machined cutting head may comprise or consist of one or more of: vibratory finishing, electrolytic polishing, blasting with dry ice, blasting with glass beads or other blasting material, or brushing directly in a CNC machine. The step of surface finishing does not comprise any of: turning; milling; grinding; hardening and tempering; and passivation.

All of the features described above in relation to the first aspect of the invention apply equally to the second aspect.

According to a third aspect, the present disclosure provides a method of manufacturing a perforator component, comprising the steps of the method according to the first or second aspects, and further comprising a step of providing a connector for reversibly coupling the cutting head to a drive shaft, the connector comprising a harder connector portion having a higher HRC, and a softer connector portion having a lower HRC.

Optionally, the method further comprises a step of joining the harder connector portion to the inner cutting head. By joining the harder connector portion to the inner cutting head, the cutting head may be directly engageable by the drive shaft of a perforator.

The harder connector portion preferably has a higher Rockwell C hardness than the cutting head.

Optionally, the harder connector portion has a HRC of at least 40 HRC while the softer connector portion has a HRC of less than 30 HRC. The softer connector portion may be a portion of, or provided on, the cutting head, which as described above, has a Rockwell C hardness of less than 30 HRC. The inventors have surprisingly found that combining a relatively softer connector portion with a relatively harder connector portion allows for the perforator to be very functionally reliable while at the same time being cheaper and quicker to manufacture (in particular if the softer connector portion is provided on the cutting head).

In typical perforator designs, the cutting head is biased away from the drive shaft with a spring, so that when there is no pressure applied to the tip of the cutting head, the cutting head and the shaft are disconnected, and the cutting head does not rotate even when the drive shaft is rotating. Connection of the cutting heads to the drive shaft is controlled using a connector, which couples the cutting head to the drive shaft when pressure is applied to the tip of the cutting head. When the pressure is removed, the connector uncouples the cutting head from the drive shaft, so that the cutting head ceases rotation. A connector portion may rotate with the drive shaft, so at the moment of contact, the connector portion of the stationary cutting head comes into contact with the connector portion of the drive shaft when it is already rotating at high speeds.

The need for the connector and the cutting head to survive this sudden impact has typically led the designers of prior art devices to make all connecting components as hard as possible. Typically both cooperating portions (both the pin and the slot, for example) are made from the same high hardness material, with the aim of avoiding damage when the two parts impact one another during use.

The present inventors have found that if the hardness of the softer connector portion, which may be provided on the cutting head, is too close to the hardness of the harder connector portion of a perforator, the parts can potentially damage one another, which can occasionally prevent safe functioning of the decoupling mechanism once trepanation is complete.

In the perforator as manufactured according to the present disclosure, due to the relatively low hardness of the softer connector portion compared to the relatively high hardness of the harder connector portion, the coupling mechanism components cooperate to smoothen the softer portion upon first coupling, thereby ensuring secure decoupling of the cutting head when it moves to the distal position.

In other words, in the perforator as manufactured according to the present disclosure, the connector is configured such that when the cutting head is in the distal position, the harder connector portion is not engaged with the softer connector portion, and when the cutting head is in the proximal position, the harder connector portion comes into contact with the softer connector portion such that the connector couples the inner cutting head to the drive shaft. The connector may also, or alternatively, couple an inner cutting head to an outer cutting head.

The term “distal” refers herein to portions of the perforator positioned towards the cutting edges of the perforator, which is intended to contact the bone tissue in use, while the term “proximal” refers to portions that are further from the bone being drilled and closer to, for example, the hand of the user holding the drill.

Optionally, the harder connector portion has a HRC of at least 45 HRC; further optionally of at least 50 HRC; and yet further optionally about 55 HRC.

Optionally, the softer connector portion is softer than the harder connector portion by at least 10 HRC; optionally by at least 20 HRC; optionally by at least 30 HRC; optionally by about 37 HRC, for example between 34 and 40 HRC. In other words, the harder connector portion may be harder than the softer connector portion by at least 10 HRC; optionally by at least 20 HRC; optionally by at least 30 HRC; optionally by about 37 HRC.

Optionally, the harder connector portion is made of stainless steel. Further optionally, the harder connector portion is made of 1.4034 stainless steel. Alternatively, the connector is made of 1.4057; 1.4021; or 1.4112 stainless steel. The harder connector portion may be hardened.

Optionally, the connector comprises: at least one coupling pin; and/or at least one ball, and/or at least one slot or calotte; and/or at least one drive surface such as an edge, an inclined plane, or a helix plane. For example, the connector may comprise at least one pin attached to the cutting head, and at least one slot for engagement with the at least one pin in a distal end of the drive shaft. Alternatively, at least one slot may be provided on the cutting head for engagement with at least one pin on the drive shaft.

Alternatively, at least one of the cutting head and the drive shaft may comprise a drive surface, configured to engage a drive surface, or a pin, on the other of the cutting head and the drive shaft. A perforator having a connector comprising a drive surface and a pin is disclosed in WO2021198418A1, and any disclosure relevant to connecting means between the drive shaft and the cutting head is incorporated herein by reference.

Pin and slot connectors are known in prior art perforators, and ball connectors are known in other fields. Advantageously, each type of connector may provide secure releasable engagement between the cutting head and the drive shaft of a perforator, or between an inner and an outer cutting head.

In known slot-and-pin type connectors of the type disclosed in U.S. Pat. No. 4,456,010, the positions of slot and pin portions may be reversed. For example, the pin may be mounted in the drive shaft with the slot in the cutting head, or vice versa.

The connector may comprise, for example, a pin mounted on the drive shaft so that the pin extends radially from the shaft. The pin is preferably configured to engage with a corresponding slot in the cutting head in order to couple the cutting head to the drive shaft. Alternatively, the connector may comprise a section of the drive shaft comprising a slot. The slot may be configured to engage with a corresponding pin mounted on the cutting head.

At least a portion of the connector, e.g. the harder connector portion, may be made up of standard parts, i.e. standard elements. Standard parts, or elements, are parts which are used in a wide range of products, and which may comply with specific DIN, or ISO, norms. They are very widely available and cheap.

In a fourth aspect, the disclosure provides a cutting head manufactured by the method according to the first or second aspects, and in a fifth aspect, the disclosure provides a perforator component manufactured by the method according to the third aspect.

In a sixth aspect, the disclosure provides a perforator for drilling bone tissue, comprising: a drive shaft with a rotational axis A; an inner cutting head with the same rotational axis A; and an outer cutting head arranged coaxially around the inner cutting head. The inner cutting head is configured to be displaceable relative to the drive shaft along the rotational axis A between a distal position, in which the cutting heads are not driveable by the drive shaft, and a proximal position, in which the cutting heads are driveable by the drive shaft. The perforator further comprises a connector for reversibly coupling at least two of the drive shaft, the inner cutting head and the outer head to one another when the inner cutting head is in the proximal position. The connector comprises a harder connector portion having a first Rockwell C hardness HRC, and a softer connector portion having a second HRC lower than the first HRC.

The connector of the perforator is made up of at least two cooperating parts—the harder connector portion and the softer connector portion-which engage with one another to couple at least two of the inner cutting head, the outer cutting head, and the drive shaft to one another. A connector may alternatively be referred to as a coupling mechanism. A variety of connector types are known in the art, such as pin-and-slot type clutches, in which the connector is made up of a pin and a separate slot that engage one another when the inner cutting head is in the proximal position.

In the prior art, both parts of the connector (i.e. both the pin and the slot) are made of the same material, which the present inventors have found can lead to problems. In the present disclosure, the inventors have found that benefits can be obtained by forming the two cooperating parts of the connector from materials having different hardnesses. For example a pin-and-slot type connector may be provided in which the pin is a harder connector portion and the slot is a softer connector portion, or vice versa.

As set out above in relation to the method of the present disclosure, providing the perforator with a relatively softer connector portion and a relatively harder connector portion allows for the perforator to be very functionally reliable while at the same time, in particular when the softer connector portion is provided on a cutting head manufactured according to the method of the present disclosure, being cheaper and quicker to manufacture.

In particular, the inventors have surprisingly found that if the hardness of the connector portions (for example the pin and the slot of a pin-and-slot connector) is too similar, the connector portions can damage one another, which can occasionally prevent safe functioning of the decoupling mechanism once trepanation is complete. Due to the relatively lower hardness of the softer connector portion compared to the relatively higher hardness of the harder connector portion, in the perforator of the present disclosure, the connector portions, or coupling mechanism components, cooperate to smoothen the connector portions upon first coupling, thereby ensuring secure decoupling of the cutting head.

The connector is configured such that when the inner cutting head is in the distal position, the harder connector portion is not engaged with the softer connector portion, and when the inner cutting head is in the proximal position, the harder connector portion comes into contact with the softer connector portion such that the connector couples, at least, two of the inner cutting head, the outer cutting head, and the drive shaft to one another. For example, the connector may couple the inner cutting head to the drive shaft, or the inner cutting head to the outer cutting head, or the outer cutting head to the drive shaft. Alternatively, the connector may couple together all three of the inner cutting head, the outer cutting head, and the drive shaft when the inner cutting head is in the proximal position.

Optionally, the harder connector portion has a first HRC higher than 30 HRC, and the softer connector portion has a second HRC lower than 30 HRC.

Optionally, the second HRC, of the softer connector portion, is less than 25 HRC. Further optionally, the second HRC is less than 20 HRC. Further optionally, the second HRC is about 18 HRC, for example between 16 and 20 HRC, or between 17 and 19 HRC, or between 17.5 and 18.5 HRC.

Optionally, the first HRC, of the harder connector portion, is at least 40 HRC. Further optionally, the first HRC is at least 45 HRC. Further optionally, the first HRC is at least 50 HRC. Yet further optionally, the first HRC is about 55 HRC, for example between 52 HRC and 58 HRC.

Optionally, the softer connector portion is softer than the harder connector portion by at least 10 HRC; optionally by at least 20 HRC; optionally by at least 30 HRC; optionally by about 37 HRC, for example between 34 and 40 HRC. In other words, the harder connector portion may be harder than the softer connector portion by at least 10 HRC; optionally by at least 20 HRC; optionally by at least 30 HRC; optionally by about 37 HRC.

As is known for connectors such as pin-and-slot type connectors, the positions of the connector portions may be reversed in the perforator without affecting the function of the perforator. For example a perforator may be provided with a slot in the distal end of the driving shaft and a cooperating pin attached to a proximal end of the inner cutting head, and when the cutting head moves to the proximal position the pin is received in the slot and the drive shaft is coupled to the cutting head. Likewise, if the positions of the pin and the slot are reversed so that the slot is provided on the proximal end of the cutting head and the pin is provided on the distal end of the drive shaft, the connector still functions in the same way, as when the cutting head moves to the proximal position the pin is received in the slot and the drive shaft is coupled to the cutting head.

In a similar way, the locations of the harder connector portion and the softer connector portion may alternate without affecting the function of the perforator of the present disclosure.

Optionally, the connector is configured to reversibly couple the drive shaft to at least one of the cutting heads when the inner cutting head is in the proximal position, so that the connector transmits rotational motion from the drive shaft to the cutting heads.

In some preferred embodiments, one of the connector portions is provided on the inner cutting head, and the other of the connector portions is provided on the drive shaft.

In some preferred embodiments, the connector is configured to reversibly couple all three of the drive shaft, the inner cutting head and the outer head to one another when the inner cutting head is in the proximal position. Advantageously, the connector coupling all three of the drive shaft, the inner cutting head and the outer head allows for fewer components to be used.

Optionally, the softer connector portion comprises a first softer connector portion provided on one of the inner cutting head, the outer cutting head and the drive shaft, and a second softer connector portion provided on another one of the inner cutting head, the outer cutting head and the drive shaft.

Optionally, the first softer connector portion is provided on the drive shaft, the second softer connector portion is provided on the outer cutting head, and the harder connector portion is provided on the inner cutting head. In particular, the first softer connector portion may be a slot in a distal end of the drive shaft, the second softer connector portion may be a triangular opening in the outer cutting head, and the harder connector portion may be a pin on the proximal end of the inner cutting head.

Optionally, the softer connector portion is provided on at least one of: the inner cutting head, the outer cutting head, and the drive shaft; and the harder connector portion is provided on another at least one of: the inner cutting head, the outer cutting head, and the drive shaft.

In some embodiments, the softer connector portion is provided on the inner cutting head. When the softer connector portion is provided on the inner cutting head, it may preferably be provided as at least one slot in the inner cutting head. The entire inner cutting head may have a HRC which is equal to the second HRC, i.e. if the softer connector portion is at least one slot in the inner cutting head, the second HRC of the softer connector portion is equal to the HRC of the inner cutting head.

The softer connector portion may alternatively be provided on the drive shaft, for example as a pin attached to the drive shaft, or a slot formed in the drive shaft. The entire drive shaft may, advantageously, have a HRC which is equal to the second HRC.

In some embodiments, one of the harder or softer connector portions is provided on the inner cutting head, and the other of the connector portions is provided on the drive shaft.

For example, if the harder connector portion is provided on the inner cutting head, the softer connector portion is provided on the drive shaft. Alternatively, if the harder connector portion is provided on the drive shaft, the softer connector portion is provided on the inner cutting head.

Optionally, the softer connector portion is provided on at least one of: the inner cutting head, the outer cutting head, and the drive shaft; and the harder connector portion is unattached to any other component of the perforator. Optionally, in this embodiment, two or all three of the inner cutting head, the outer cutting head, and the drive shaft may be provided with a softer connector portion. In this embodiment, the harder connector portion may be at least one ball. The perforator may be configured so that the ball is free to move relative to the inner cutting head and the outer cutting head when the inner cutting head is in the distal position, and configured so that the ball is confined between the inner cutting head and the outer cutting head when the inner cutting head is in the proximal position.

A variety of connector types may be used in this aspect, as long as the connector comprises a softer connector portion configured to engage with a harder connector portion when the inner cutting head is in the proximal position.

Optionally, the connector comprises: at least one coupling pin; and/or at least one ball; and/or at least one slot, notch or calotte (part-spherical cavity); and/or at least one drive surface such as an edge, an inclined plane, a helix plane. For example the connector may comprise at least one coupling pin and at least one slot, or the connector may comprise at least one ball and at least one slot or cavity for receiving the ball when the cutting head is in the proximal position. Slots may be square sided, semi-cylindrical, or preferably shaped with an inclined lead-in edge and a drive surface for urging rotation of the pin or ball when they are engaged in the slot.

Optionally, a portion of the connector may be configured to transform rotation of the inner and outer cutting heads relative to one another around the rotational axis A into translational displacement of the inner cutting head along the rotational axis. For example, the perforator may comprise softer and harder connector portions configured to transmit the rotation of the drive shaft to the outer cutting head when the inner cutting head is in the proximal position.

Optionally, one of the harder and softer connector portions comprises an angled edge configured to transform relative rotation of the inner and outer cutting heads into translational displacement of the inner cutting head along the rotational axis. Alternatively, the harder connector portion is a, or the, coupling pin, and the outer cutting head comprises, in a wall, a groove or opening, the groove or opening having a proximal end for receiving the pin when the inner cutting head is in the proximal position, and an angled edge for guiding the pin towards the distal position in response to relative rotation of the inner and outer cutting heads.

Optionally, the inner cutting head is made of stainless steel. Further optionally, the stainless steel is austenitic. Yet further optionally, the stainless steel is one of: 1.4301; 1.4305; 1.4306; 1.4307; 1.4310; 1.4401; 1.4404; 1.4435; and 1.4441. In a preferred embodiment of the perforator, the inner cutting head is made of unhardened 1.4404 stainless steel. This embodiment is particularly preferred if the softer connector portion is provided on the inner cutting head, e.g. as a slot.

Optionally, the inner cutting head comprises cutting edges which are machined. Optionally, the cutting edges are drilled and milled. Further optionally, the cutting edges are CNC drilled and milled.

Optionally, the cutting edges are surface treated, or surface finished. Optionally, the cutting edges are surface treated by: finishing; and/or polishing. Further optionally, the cutting edges are vibratory finished and/or electrolytically polished.

According to a seventh aspect of the present disclosure, there is provided a perforator for drilling bone tissue, comprising: a drive shaft with a rotational axis A; an inner cutting head with the same rotational axis A; and a connector for reversibly coupling the inner cutting head to the drive shaft, in which the inner cutting head is configured to be displaceable relative to the drive shaft along the rotational axis A between a distal position, in which the inner cutting head is not driveable by the drive shaft, and a proximal position, in which the connector couples the inner cutting head to the drive shaft to transmit rotational motion from the drive shaft to the inner cutting head, and in which the connector comprises a harder connector portion having a first Rockwell C hardness HRC, and a softer connector portion having a second HRC lower than the first HRC, wherein the soft connector portion is provided on at least one of the inner cutting head and the drive shaft.

Optionally, the perforator further comprises an outer cutting head arranged coaxially around the inner cutting head. The outer cutting head may preferably be made from the same material as the inner cutting head, so that a Rockwell C hardness of the outer cutting head may be less than 30 HRC. The outer cutting head may preferably be a cutting head manufactured according to an aspect of the disclosure described above.

In embodiments comprising an outer cutting head, the connector may further be configured to couple the outer cutting head to the inner cutting head when the inner cutting head is in the proximal position, so that rotational motion is transmitted from the inner cutting head to the outer cutting head. Optionally one, or a portion of one, of the softer or harder connector portions may be provided on the outer cutting head.

According to an eighth aspect of the present disclosure, there is provided a perforator for drilling bone tissue, comprising: a drive shaft with a rotational axis A; an inner cutting head with the same rotational axis A; and an outer cutting head arranged coaxially around the inner cutting head. The inner cutting head is configured to be displaceable relative to the drive shaft along the rotational axis A between a distal position, in which the cutting heads are not driveable by the drive shaft, and a proximal position, in which the cutting heads are driveable by the drive shaft. The perforator further comprises a connector for reversibly coupling the inner cutting head and the outer head to one another when the inner cutting head is in the proximal position, and in which the connector comprises a harder connector portion having a first Rockwell C hardness HRC, and a softer connector portion having a second HRC lower than the first HRC.

According to a ninth aspect of the present disclosure, there is provided a cutting head for a perforator for cutting bone tissue, the cutting head comprising a plurality of cutting edges, characterised in that the cutting head consists of austenitic steel having a Rockwell C hardness HRC of less than 30 HRC.

The austenitic steel may be one of: 1.4301; 1.4305; 1.4306; 1.4307; 1.4310; 1.4401; 1.4404; 1.4435; and 1.4441.

The austenitic steel may have a HRC of less than 25 HRC, or less than 20 HRC, or of between 16 and 20 HRC, or between 17 and 19 HRC, or between 17.5 and 18.5 HRC. In one particular example, the austenitic steel has a HRC of about 18 HRC.

The “perforator for cutting bone tissue” may be referred to as a “bone perforator”, a “cranial perforator”, or a “cranial drill”. The cutting head for a perforator may be referred to as a “perforator cutting head”, a “bone perforator cutting head”, a “cranial perforator cutting head”, or a “cranial drill cutting head”.

Any feature in one aspect of the disclosure may be applied to other aspects of the disclosure, in any appropriate combination. Furthermore, any, some and/or all features in one aspect may be applied to any, some and/or all features in any other aspect, in any appropriate combination. In particular, any method features provided in relation to the first aspect may be applied to any of the other aspects. Further, and in particular, any feature provided in relation to the perforator according to the sixth aspect may, mutatis mutandis, be applied to a perforator according to the seventh aspect or the eighth aspect or the cutting head according to the ninth aspect.

It should also be appreciated that particular combinations of the various features described and defined in any aspects of the invention may be implemented and/or supplied and/or used independently.

### DESCRIPTION OF SPECIFIC EMBODIMENTS OF THE INVENTION

Specific embodiments of the invention will now be described with reference to the figures, in which:

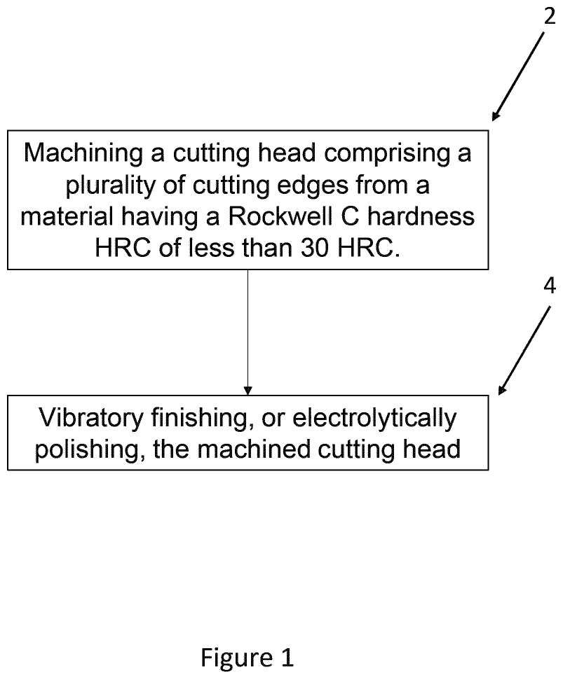

FIG. 1 is a flow diagram of an exemplary method according to the disclosure;

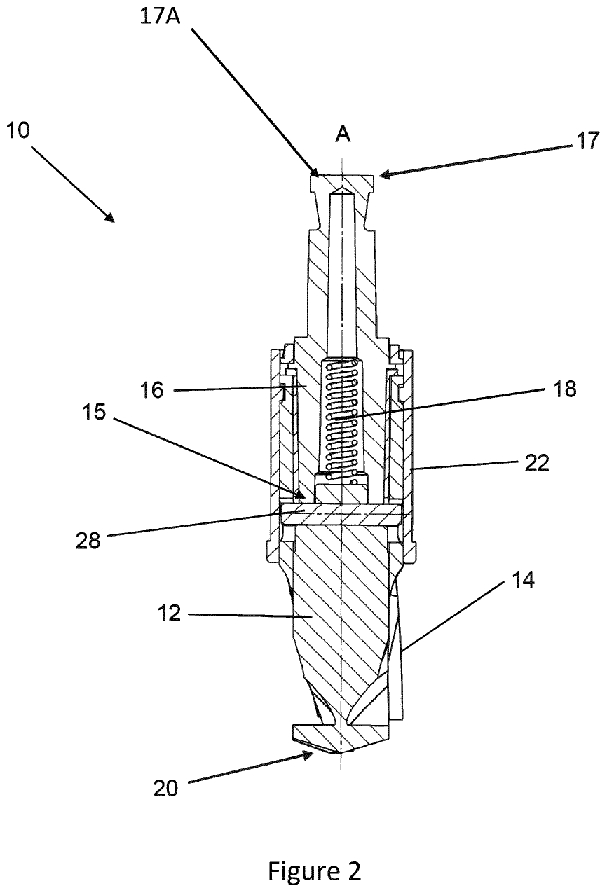

FIG. 2 is a cross-section taken along the rotational axis A of an exemplary perforator according to the disclosure;

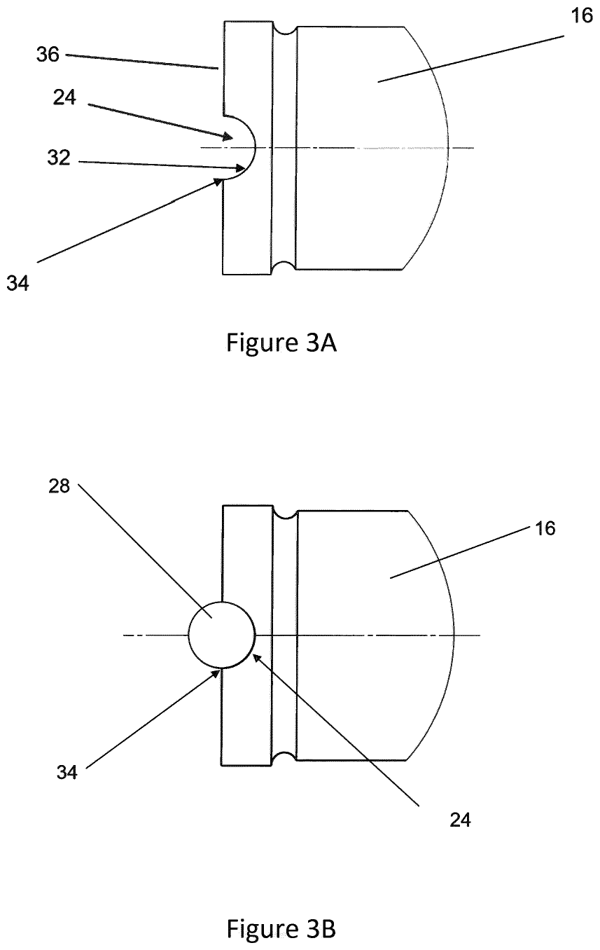

FIG. 3a is a side-on view of the distal end of an exemplary drive shaft;

FIG. 3b is a close-up side-on view of the exemplary drive shaft of FIG. 3a engaged with a connector pin;

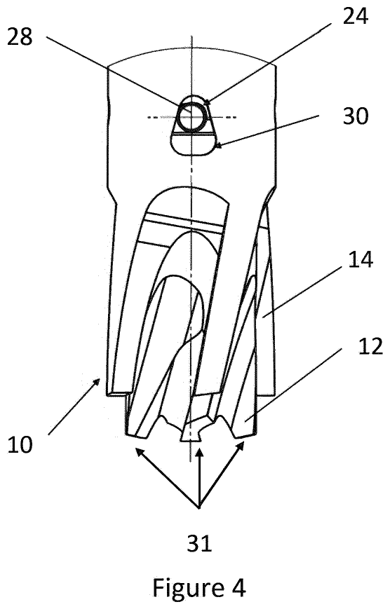

FIG. 4 is a semi-transparent view of the assembled exemplary perforator without its cylindrical housing in position;

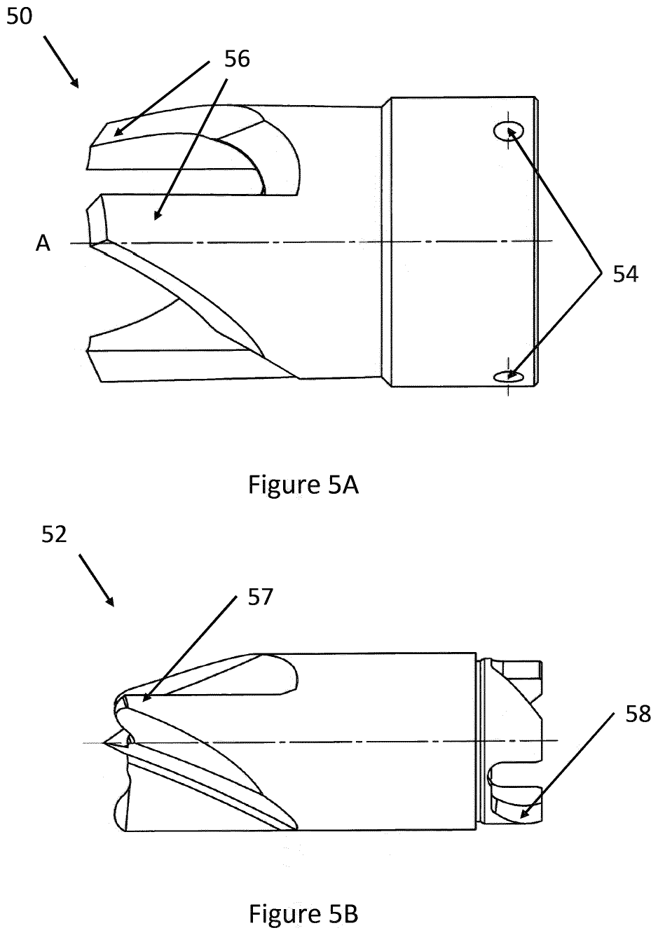

FIG. 5a is a side-on view of an outer cutting head of a further exemplary perforator according to the present disclosure;

FIG. 5b is a side-on view of an inner cutting head of the further exemplary perforator of FIG. 5a;

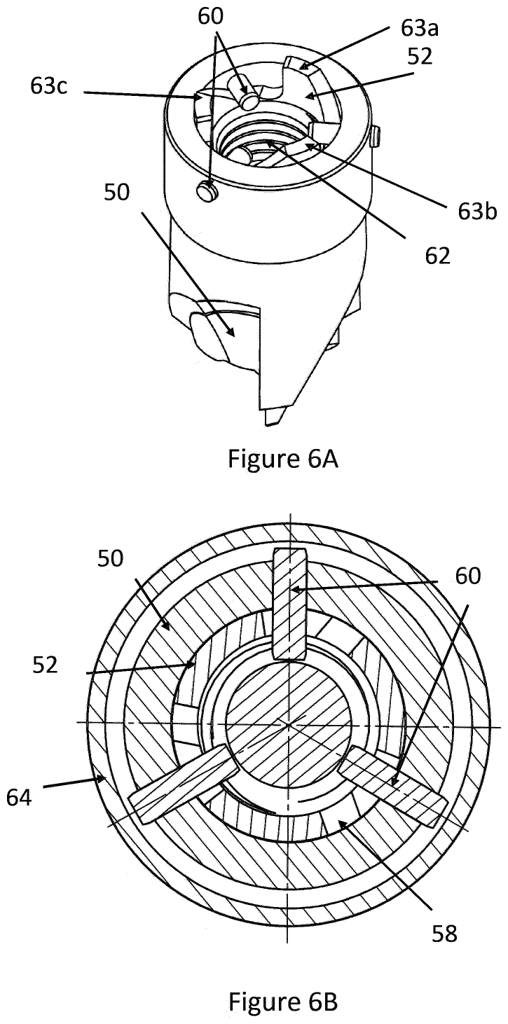

FIG. 6a is a perspective isometric view of a cutting head assembly of the further exemplary perforator of FIGS. 5a and 5b;

FIG. 6b is a cross sectional view of the further exemplary perforator of FIGS. 5a, 5b, and 6a;

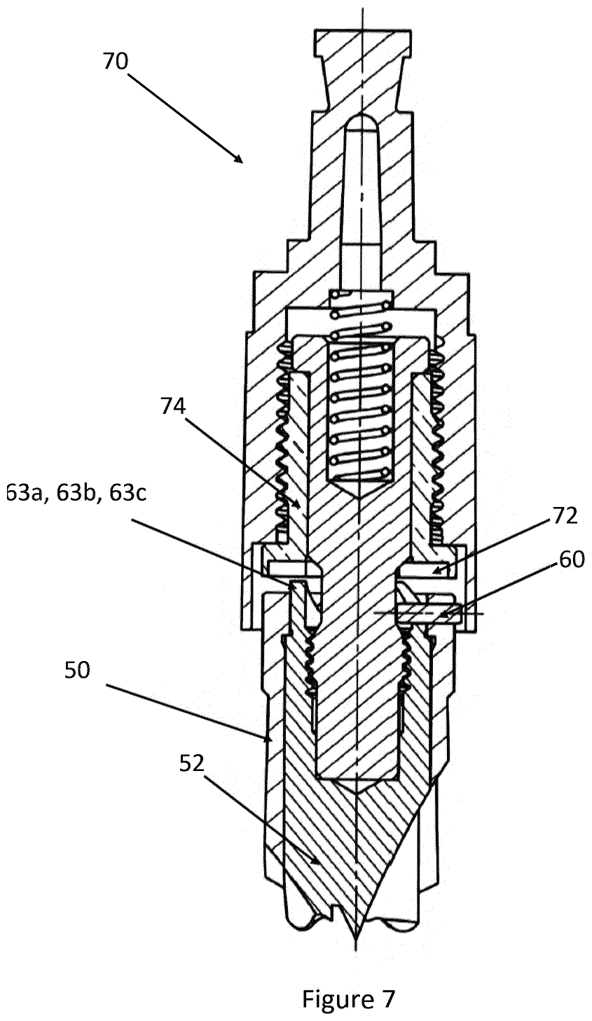

FIG. 7 is a cross section taken along the rotational axis A of the further exemplary perforator of FIGS. 5a, 5b, 6a, and 6b;

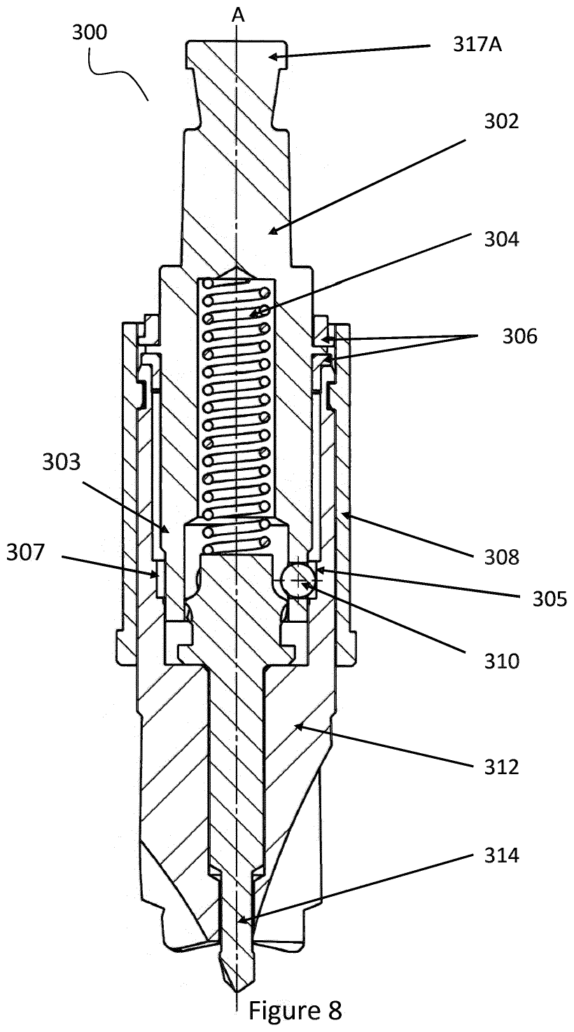

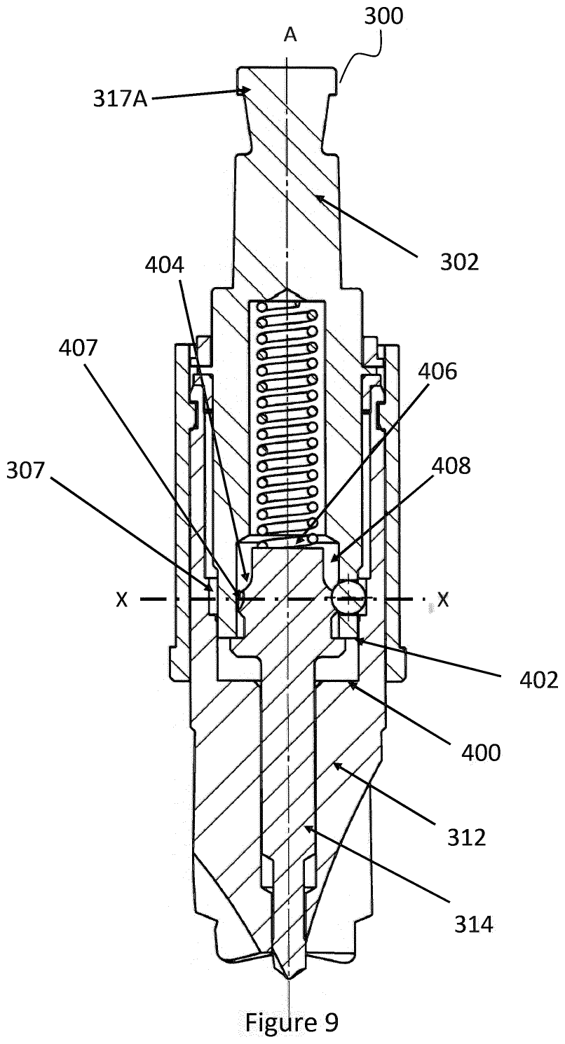

FIGS. 8 and 9 are cross sections taken along the rotational axis A of a further exemplary perforator, with the inner cutting head in a distal position and a proximal cutting position, respectively;

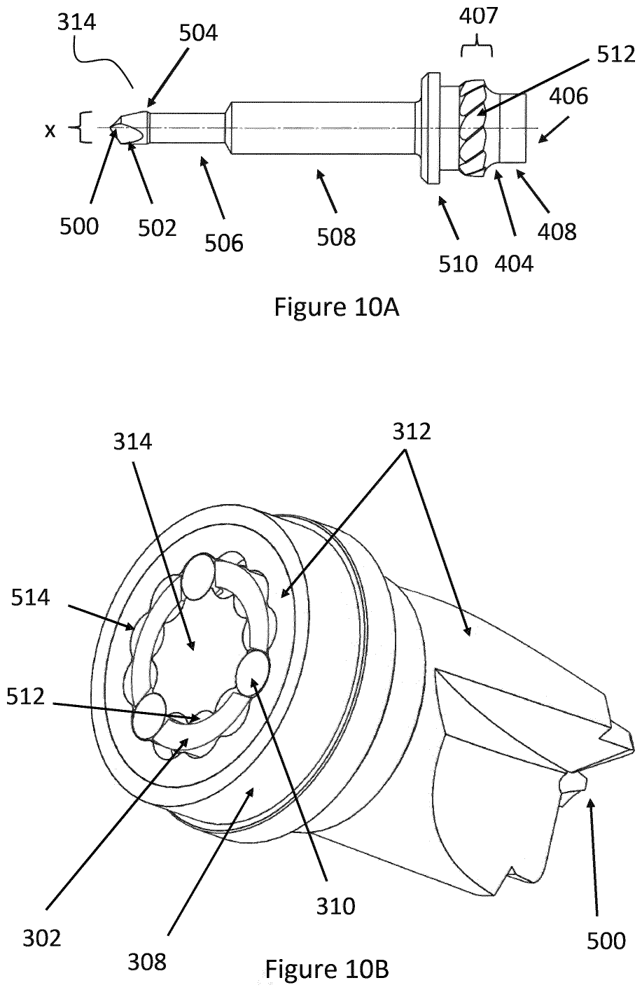

FIG. 10a is a side view of an inner cutting of the further exemplary perforator of FIGS. 8 and 9; and

FIG. 10b is a perspective isometric cross section of the further exemplary perforator of FIGS. 8, 9, and 10a.

### SPECIFIC DESCRIPTION

FIG. 1 illustrates the steps of a method according to the present disclosure, including a step of machining a cutting head comprising a plurality of cutting edges from a material having a Rockwell C hardness HRC of less than 30 HRC. In an exemplary embodiment, the material is unhardened stainless steel 1.4404 having a HRC of about 18 HRC (equivalent to about 215 HB).

A blank of the starting material (stainless steel 1.4404) having a HRC of less than 30 is set up in a CNC machining apparatus, and machined in a single CNC machining setup by drilling and milling the blank into the shape of a perforator cutting head. The cutting head is machined directly to its desired finished shape, and the method only includes a further step of superficially finishing, or treating, the machined cutting head.

The shape of a perforator cutting head is well known in the art, and comprises a plurality of cutting edges configured to cut bone when the cutting head is used in a perforator.

In the exemplary embodiment of the method, the superficial finishing step consists of vibratory finishing.

In the exemplary embodiment, a OTEC CF-Serie vibratory finisher is used with ceramic abrasive elements. The shape of the abrasive elements may differ from round to sharp-edged, however in the exemplary embodiments are triangular prisms having a height of 1 to 5 mm and a tip to tip diameter of 1 to 5 mm.

It is noted that larger abrasive elements are more abrasive, and could damage thin-walled parts. As such, the size of the abrasive elements must be selected so that they are appropriately sized.

In the exemplary embodiment, the vibration speed is set to 220 to 350 rpm for rounding edges, and to 120 to 220 rpm for smoothing the surface. A cutting oil is used as lubricant, although other emulsions may be used. The cutting oil is the same cutting oil used in the CNC drilling and milling. This advantageously facilitates cleaning of the parts, and the oil protects the parts during vibratory finishing.

In the exemplary embodiment about 50 to 200 cutting heads, e.g. 125 cutting heads, are introduced into the vibratory finisher at the same time. The more parts are placed in the vibratory finisher at the same time, the higher the abrasion. The fewer parts are placed in the vibratory finisher, the longer the time period for which vibratory finishing must be carried out.

Following vibratory finishing, the cutting head is ready for use in a perforator without any further processing steps.

The superficial finishing step may alternatively consist of electrolytically polishing the machined cutting head.

Unlike the prior art, the method does not comprise any steps of grinding, hardening or tempering the cutting head.

As the method does not involve hardening or tempering of the starting material, the hardness of the cutting head is substantially the same as the hardness of the starting material used for the blank. Thus in an exemplary embodiment where the starting material is unhardened stainless steel 1.4404 having a HRC of about 18 HRC, the cutting head produced by the method also has a hardness of about 18 HRC.

FIGS. 2 to 4 show a perforator design similar to the prior art perforator disclosed in U.S. Pat. No. 4,456,010. However, in the perforator according to the present disclosure, the cutting heads of said perforator have a hardness of less than 30 HRC, and preferably about 18 HRC, while the connector pin has a hardness of about 55 HRC, being made of hardened stainless steel 1.4034.

In FIG. 2, the perforator 10 comprises an inner cutting head 12, a hollow outer cutting head 14 arranged coaxially around the inner cutting head, and a drive shaft 16, all rotatable about the same axis of rotation A. The drive shaft has a distal end 15 and a proximal end 17. The proximal end 17 is connectable to a hand-held drill housing a motor, for example. In particular, the proximal end 17 is a standardised connector 17A, such as a Hudson connector.

The distal end of the perforator 10 terminates in the distal tip 20 of the inner cutting head, which contacts bone in use and is shown at the bottom of FIG. 2. The proximal end of the perforator 10 terminates in the standardised connector 17A of the drive shaft, which is shown at the top in FIG. 2.

The inner cutting head 12 is movable along the rotational axis A between two positions: a distal position, in which the inner cutting head is not connected to the drive shaft 16, and a proximal cutting position, in which the inner cutting head 12 is connected to the drive shaft 16. The inner cutting head is biased away from the drive shaft 16 and into the distal position by a spring 18, so that the inner cutting head only moves into the proximal cutting position when the distal tip 20 of the inner cutting head 12 is pressed against a surface, such as bone to be drilled. The force with which the distal tip 20 has to be pressed against the surface to overcome the biasing force of the spring 18 is determined by the spring constant of the spring 18 and the axial separation between the distal position and the proximal cutting position.

When no pressure is applied to the distal tip 20 of the inner cutting head 12, the inner cutting head 12 and the drive shaft 16 are disconnected, and neither of the inner or outer cutting heads 12, 14 rotates even when the drive shaft 16 is rotating. A cylindrical housing 22 is arranged coaxially around the outer cutting head 14.

Connection of the inner cutting head 12 and the drive shaft 16 is achieved using a slot-and-pin type clutch that comprises a slot 24 (not visible in FIG. 2, as the cross-section is taken along the slot, but shown in FIGS. 3A and 3B) in the distal end 15 of the drive shaft 16, and a corresponding clutch-pin 28 which extends diametrically through the inner cutting head 12 near its proximal end. The clutch-pin 28 and slot 24 acts as a connector that reversibly couples the inner cutting head with the drive shaft. At least the slot 24, but optionally the distal end 15 of the drive shaft or the entire drive shaft 16, has a lower HRC hardness than the clutch-pin 28. In particular, the HRC hardness of the slot 24 (or the distal end 15, or the drive shaft 16) may be less than 30 HRC, or about 18 HRC.

In other words, in this embodiment, the connector which reversibly couples the inner cutting head 12 to the drive shaft 16 comprises a harder portion being a clutch-pin 28 provided on the inner cutting head 12, and a softer portion being a slot 24 provided in the distal end 15 of the drive shaft 16.

The outer cutting head 14 is not translatable along the rotational axis A, and comprises two triangular-shaped openings 30 through which the two opposite ends of the clutch-pin 28 extend, as shown in FIG. 4a. The triangular openings 30 are arranged so that the apex of each triangular opening 30 points towards the proximal end of the perforator. Thus, translation of the inner cutting head 12 towards the proximal cutting position moves the clutch-pin 28 towards the apex of the triangular opening 30.

As the distal tip 20 of the inner cutting head 12 is pressed against bone in use, a force is applied which moves the inner cutting head 12 against the biasing spring 18 until the clutch-pin 28 engages with the slot 24 in the rotating drive shaft 16. In this proximal cutting position, the clutch-pin 28 also engages with the side or apex of the triangular openings 30, so that rotational force is transmitted from the inner cutting head 12 to the outer cutting head 14, and both cutting heads rotate together. As long as force is applied to the tip 20 of the inner cutting head to keep the clutch-pin 28 engaged with the slot 24, the rotating drive shaft transmits rotational force to the cutting heads so that both cutting heads rotate and the perforator drills through the bone at an inner cutting diameter of the inner cutting head 12 and at an outer cutting diameter of the outer cutting head 14.

As soon as the distal tip 20 of the inner cutting head 12 perforates the bone (for example the inner surface of the cranium), however, the force applied to the inner cutting head 12 by the bone is greatly reduced, so that it is exceeded by the force exerted by the biasing spring 18 on the inner cutting head 12, so that the biasing spring 18 urges the clutch-pin 28 out of the slot 24 in the drive shaft. Both the inner and outer cutting heads should then cease to rotate immediately to prevent damage to the dura mater as the biasing spring 18 urges the inner cutting head 12 into its distal position.

In a preferred embodiment of the present invention, the inner cutting head 12 of the perforator 10 has a hardness of about 18 HRC, whereas the clutch-pin 28 has a hardness of about 55 HRC (+/−3 HRC). The outer cutting head 14 preferably also has a hardness of about 18 HRC. As such, in the present embodiment, the connector comprises two softer connector portions: the slot 24 in the drive shaft 16 and the triangular-shaped openings 30 of the outer cutting head 14.

An alternative perforator 70 according to the present disclosure is shown in FIGS. 5A, 5B, 6A, 6B, and 7. The perforator 70 comprises an outer cutting head 50 extending along rotational axis A, comprising apertures 54 in its proximal end for receiving connector pins 60 of the perforator. The outer cutting head 50 has cutting edges 56 at its distal end.

The perforator 70 further comprises an inner cutting head 52 having cutting edges 57 at its distal end, and engagement slots 58 for engaging with the connector pins 60.

The inner and outer cutting heads 50, 52 of the perforator 70 have a hardness of about 18 HRC, whereas the connector pins 60 have a hardness of about 55 HRC (+/−3 HRC) and are standard parts.

As described above in relation to the perforator 10 of FIGS. 2, 3a, 3b, and 4, the inner cutting head 50 of perforator 70 is displaceable along the rotational axis A between a distal position and a proximal position. In the distal position, the connector pins 60 do not engage the slots 58 in the inner cutting head 52, so that the inner cutting head 52 and the outer coupling head 50 are not coupled.

In the proximal position, the connector pins 60 engage the slots 58 in the inner cutting head 52, and the apertures 54 in the outer cutting head 50, as shown in FIG. 6B, so that rotation of the inner cutting head 52 is transmitted to the outer cutting head 50.

Between the slots 58 for engaging with the connector pins 60, i.e. forming the proximal end of the inner cutting head 52, are projections 63a, 63b, 63c, which, as shown in FIG. 6A, project beyond the distal end of the outer cutting head 50 when the inner cutting head 52 is in the proximal position.

When the inner cutting head 52 is in the proximal position, the projections 63a, 63b, 63c are configured to engage with corresponding slots 72 in a distal end of the drive shaft 74. As such, when the inner cutting head 52 is translated from the distal position to the proximal position, the projections 63a, 63b, 63c engage the corresponding slots 72 of the drive shaft 74, so that rotational motion of the drive shaft 74 is transmitted to the inner cutting head 52. As set out above, in this embodiment, the rotational motion of the inner cutting head 52 is transmitted to the outer cutting head 50 via the connector pins 60.

In this embodiment, the connector has a harder connector portion (pins 60) and a softer connector portion (slots 58), which are configured to couple the inner cutting head 52 to the outer cutting head 50.

FIGS. 8, 9, 10A, and 10B illustrate a further alternative perforator 300 according to the present disclosure. In this embodiment, the perforator 300 of the disclosure comprises a ball clutch. A hollow distal end portion 303 of the drive shaft 302 of the perforator 300 comprises three openings 305 (only one shown in FIGS. 8 and 9) provided in a cylindrical wall of the drive shaft 302. The three openings 305 are round holes and are spaced evenly around the circumference of the drive shaft. A ball 310 is held in each one of the three openings 305. Each opening 305 is sized to restrict axial movement of the ball (along the rotation axis A) and rotational movement (around the axis of rotation A) relative to the drive shaft 302.

The perforator 300 has an inner perforator pin 314, or an inner cutting head, which extends along the rotational axis A, and a cutting head 312 which is arranged coaxially around the perforator pin 314. Although the inner, translatable perforator head 314 of the perforator 300 is shown as a perforator pin 314 having a relative tip diameter significantly smaller than the cutting head 312, the perforator 300 is equally applicable to alternative perforators having an inner cutting head and an outer cutting head, or an inner drill head and outer chipping head, as described in detail above in relation to the other perforator embodiments.

The spring 304 biases perforator pin 314 away from drive shaft 302 into a distal position, as shown in FIG. 8. The drive shaft 302 engages with a cylindrical housing 308 of the perforator 300 via two sliding rings 306. The hollow distal end portion 303 of the drive shaft 302 is arranged coaxially around a proximal end of the perforator pin 314. The cutting head 312 is arranged coaxially around both the perforator pin 314 and the distal end portion 303 of the drive shaft 302. As further shown in FIG. 8, the or each ball 310 is not only confined by the opening 305 in the drive shaft 302, but also by the perforator pin 314 and cutting head 312. As such, each ball 310 is confined in a predefined space when the perforator pin 314 is in the distal position as shown in FIG. 8. Within this space, the or each ball 310 may move a radial direction (i.e. in a direction towards and away from the rotational axis A, along the radius of the perforator). As such, as at least a part of the inner surface of the cutting head 312 is radially spaced apart from the drive shaft 302, even where the inner surface of the cutting head 312 is not adjacent an opening 305 in the wall of the drive shaft 302, there may be a space 307 between the drive shaft 302 and the cutting head 312.

Each ball 310 is free to move in a circumferential direction (i.e. to rotate around the rotational axis A), e.g. in response to rotational motion of the drive shaft 302. As each ball 310 is confined so that movement relative to the drive shaft 302 is restricted, the balls 310 rotate around the rotational axis A together with the drive shaft 302. In other words, the balls 310 are free to rotate relative to the cutting head 312 and the perforator pin 314 when the perforator pin 314 is in the distal position as shown in FIG. 8. Therefore, rotational motion of the drive shaft 302 is not transmitted to the cutting head 312 or perforator pin 314 when the perforator pin 314 is in its distal position.

The perforator pin 314, similarly to the inner cutting heads 12, 52 of the prior art perforators 10, 70, is movable along the rotational axis A between two positions. In a distal position, the perforator pin 314 is not connected to the drive shaft 302 via the balls 310, and in a proximal cutting position, as shown in FIG. 9, the perforator pin 314 is connected to the drive shaft 302 via the or each ball 310. The perforator pin 314 is biased away from the drive shaft 302 and into the distal position by a biasing spring 304, so that the perforator pin 314 only moves into the proximal cutting position when the distal tip 500 of the perforator pin 314 is pressed against a surface, such as the bone to be drilled, with sufficient force.

When there is no pressure applied to the distal tip 500 of the perforator pin 314, the perforator pin 314 and cutting head 312 are disconnected from the drive shaft 302 as the ball 310 is not connected to either of the perforator pin 314 and the cutting head 312 and free to rotate relative to the cutting head 312 and the perforator pin 314 along with the drive shaft 302. Therefore, even when the drive shaft 302 is rotating, neither of the perforator pin 314 and the cutting head 312 rotate.

The perforator pin 314 terminates in a distal portion. The distal portion includes a distal head 502 having a sharpened distal tip 500. The distal tip 500 is configured to penetrate the centre of a disc of bone being cut. However, the distal head 502 and its sharpened distal tip 500 are configured to grate or scrape bone when the perforator is in use rather than to cut it. The perforator pin is also pointed to penetrate, or drill, a single continuous hole, rather than to cut around the perimeter of a circular disc of bone. As such, the function and structure of the perforator pin 314 of the present invention differs significantly from the inner cutting/drill head 12 of the prior art perforators described above.

The distal portion of the perforator pin 314 is further made up of a distal shaft 506, which has a smaller diameter than the distal head 502. As such, the proximal end of the distal head 502 connects to the distal shaft 506 forming a lip 504. The lip 504 may hold a disc of bone being cut, which has been penetrated by the distal head 502, in place. As such, the function of the lip 502 may be comparable to the barbs of a barbed arrowhead. This may ensure that a disc of bone being cut is stable during trepanation. Additionally, the lip 504 may restrict movement of the disc of bone relative to the perforator pin 314, so that once trepanation is completed, the disc of bone can be removed with the perforator 300.

The distal shaft 506 terminates proximally in a central shaft 508 having a diameter which is larger than the diameter of the distal shaft 506. The perforator pin 314, at a proximal end of the central shaft 508, has a flange 510 which is configured to engage with a flat inner surface 400 of an inner bore of the cutting head 312 upon the perforator pin 314 being in the distal position shown in FIG. 8. The flat inner surface 400 is perpendicular to the rotational axis A. As such, the axial position of the flange 510 limits axial movement of the perforator pin 314 distally. Upon the perforator pin 314 being in the proximal cutting position, as shown in FIG. 9, the flange 510 is configured to engage with a flat end surface 402 of the drive shaft 302. As such, the axial position of the flange 510 limits axial movement of the perforator pin 314 proximally.

As shown in FIGS. 10A and 10B, an engagement region 407 of the perforator pin 314 comprises a first plurality of notches 512 configured to receive a portion of the balls 310 when the perforator pin 314 is in its proximal cutting position. Similarly, an inner surface of the outer cutting head 312 comprises a second plurality of notches 514, each of which is configured to receive a portion of the balls 310.

The first plurality of notches 512 are angled relative to the rotational axis A, such that any biasing force of the spring 304, which acts upon the flat end surface 406 of the perforator pin 314 to urge it into a distal direction along rotational axis A, may be distributed between the spring 304 and the angled notches 512 coupled to one of the balls 310.

When pressure is exerted on the tip of the perforator pin 314, and the perforator pin 314 is urged from the distal position into the proximal cutting position, a cylindrical surface 408 allows for the balls 310 to easily run along the cylindrical surface 408 and into engagement with the engagement region 407 of the perforator pin 314, as shown in FIG. 9. The engagement region 407 is positioned on the perforator pin 314, on a distal side of the cylindrical surface 408, so that, as the perforator pin 314 moves into the proximal cutting position, the engagement region 407 becomes axially aligned with (i.e. positioned at the same axial height as) the openings 305 in the wall of the drive shaft 302 and the balls 310, as shown in FIGS. 9 and 10B.

At least the perforator pin 314 of the perforator 300 has a hardness of about 18 HRC, and the balls 310 have a hardness of 55 HRC (+/−3). The outer cutting head 312 may also have a hardness of about 18 HRC. In other words, the balls 310 form the harder connector portion, and at least the engagement region 407 of the perforator pin 314 forms the softer connector portion. However, in a preferred embodiment, the outer cutting head 312 and the drive shaft 302 have a hardness of 18 HRC, so that the harder connector portion, i.e. the balls 310, engage with softer connector portions on the perforator pin 314, the outer cutting head 312, and the drive shaft 302.

Although perforator 300 is shown as having a perforator pin 314 rather than an inner cutting head 12, 52, the “ball clutch” connector mechanism may equally be applied to a perforator having an inner cutting head and an outer cutting head, such as perforators 10 and 70.

The method of manufacturing of the present disclosure is applicable to the manufacturing of different types of cutting heads (inner cutting heads 12, 52, 314 or outer cutting heads 14, 50, 312), different shapes of perforator heads (e.g. the perforator pin 314 and the inner cutting heads 12, 52 and outer cutting heads 14, 50, 312), and perforators 10, 70, 300 having different connector, or clutch, mechanisms. Similarly, perforators 10, 70, 300 having any type of design and any type of connector, or clutch, mechanism may be constructed according to the present disclosure.

Embodiments of the disclosure can be described with reference to the following numbered clauses, with preferred features laid out in the dependent clauses:
1. A method of manufacturing a cutting head for a perforator, the method consisting of:
   machining a cutting head comprising a plurality of cutting edges from a
   material having a Rockwell C hardness HRC of less than 30 HRC; and
   surface treating the machined cutting head.
2. The method of clause 1, in which the cutting head is machined by drilling and milling, optionally computer numerical control CNC milling.
3. The method of clause 1 or 2, wherein the cutting head and the cutting edges are machined to their final shape in a single machining step, using a single setup.
4. The method of clause 1, 2 or 3, wherein the step of surface treating the machined cutting head comprises finishing or polishing the machined cutting head.
5. The method of any preceding clause, wherein the step of surface treating the machined cutting head comprises one of: vibratory finishing; and electrolytic polishing.
6. The method of any preceding clause, wherein the material has a HRC of less than 25 HRC, preferably between 17 and 19 HRC.
7. The method of any of the preceding clauses, wherein the material is a stainless steel, and optionally wherein the stainless steel is one of: 1.4301; 1.4305; 1.4306; 1.4307; 1.4310; 1.4401; 1.4404; 1.4435; and 1.4441.
8. A method of manufacturing a perforator component, comprising the steps of any of the preceding clauses, and further comprising a step of providing a connector for reversibly coupling the cutting head to a drive shaft, the connector comprising a harder connector portion having a higher HRC, and a softer connector portion having a lower HRC.
9. The method of clause 8, further comprising a step of joining the harder connector portion to the inner cutting head.
10. The method of clause 8 or 9, wherein the harder connector portion has a HRC of at least 40 HRC.
11. The method of clause 8, 9, or 10, wherein the connector comprises: at least one coupling pin; and/or at least one ball; and/or at least one slot or calotte; and/or at least one drive surface such as an edge, an inclined plane, a helix plane.
12. A cutting head manufactured by the method of any of clause 1 to 7, or a perforator component manufactured by the method of any of clause 8 to 11.
13. A perforator for drilling bone tissue, comprising: a drive shaft with a rotational axis A; an inner cutting head with the same rotational axis A; and an outer cutting head arranged coaxially around the inner cutting head, in which the inner cutting head is configured to be displaceable relative to the drive shaft along the rotational axis A between a distal position, in which the cutting heads are not driveable by the drive shaft, and a proximal position, in which the cutting heads are driveable by the drive shaft, in which the perforator comprises a connector for reversibly coupling at least two of the drive shaft, the inner cutting head and the outer head to one another when the inner cutting head is in the proximal position, and in which the connector comprises a harder connector portion having a first Rockwell C hardness HRC, and a softer connector portion having a second HRC lower than the first HRC.
14. The perforator of clause 13, in which the first HRC, of the harder connector portion, is higher than 30 HRC, and the second HRC, of the softer connector portion, is lower than 30 HRC.
15. The perforator of clause 13 or 14, wherein the second HRC, of the soft connector portion, is less than 25 HRC, optionally the second HRC is about 18 HRC.
16. The perforator of clause 13, 14 or 15, wherein the first HRC, of the harder connector portion, is at least 40 HRC.
17. The perforator of any of clauses 13 to 16, wherein the softer connector portion is softer than the harder connector portion by at least 10 HRC, or at least 20 HRC, or at least 30 HRC.
18. The perforator of any of clauses 13 to 17, in which the connector is configured to reversibly couple the drive shaft to at least one of the cutting heads when the inner cutting head is in the proximal position, so that the connector transmits rotational motion from the drive shaft to the cutting heads.
19. The perforator of clause 18, in which one of the connector portions is provided on the inner cutting head, and the other of the connector portions is provided on the drive shaft.
20. The perforator of any of clauses 13 to 18, wherein the connector is configured to reversibly couple all three of the drive shaft, the inner cutting head and the outer head to one another when the inner cutting head is in the proximal position.
21. The perforator of clause 20, in which the softer connector portion comprises a first softer connector portion provided on one of the inner cutting head, the outer cutting head and the drive shaft, and a second softer connector portion provided on another one of the inner cutting head, the outer cutting head and the drive shaft.
22. The perforator of any of clause 20 or 21, wherein the softer connector portion is provided on at least one of: the inner cutting head, the outer cutting head, and the drive shaft; and the harder connector portion is provided on another at least one of: the inner cutting head, the outer cutting head, and the drive shaft.
23. The perforator of any of clause 20 or 21, wherein the softer connector portion is provided on at least one of: the inner cutting head, the outer cutting head, and the drive shaft; and the harder connector portion is unattached to any other component of the perforator.
24. A perforator according to any of clause 13 to 17, in which the connector is configured to reversibly couple the inner cutting head to the outer cutting head when the inner cutting head is in the proximal position, so that the connector transmits rotational motion from the inner cutting head to the outer cutting head.
25. A perforator according to clause 24, in which one of the connector portions is provided on the inner cutting head, and the other of the connector portions is provided on the outer cutting head.
26. The perforator of clause 24 or 25, further comprising a drive shaft connector for reversibly coupling the drive shaft to at least one of the cutting heads when the inner cutting head is in the proximal position.
27. A method of manufacturing a cutting head for a perforator, the method comprising the steps of: machining a cutting head comprising a plurality of cutting edges from a material having a Rockwell C hardness HRC of less than 30 HRC; and surface finishing the machined cutting head, in which the method does not comprise steps of hardening or grinding the machined cutting head.
28. The method of clause 27, comprising only the steps of: machining the cutting head from a material having a Rockwell C hardness HRC of less than 30 HRC, and surface finishing the machined cutting head.
29. The method of clause 27 or 28, wherein the step of machining the cutting head from the starting material comprises one or more mechanical machining processes, optionally wherein the step of machining comprises, or consists of, one or more of drilling, milling, single-point cutting or multi-point cutting.
30. The method of clause 29, wherein the cutting head is machined from a blank of the starting material by drilling and/or milling, and optionally wherein the machining is by computer numerical control CNC drilling and milling.
31. The method of any of clauses 27 to 30, wherein the step of surface finishing the machined cutting head comprises finishing or polishing the cutting head and/or the cutting edges, and optionally wherein the step of surface finishing the machined cutting head comprises, or consist of, one or more of: vibratory finishing, electrolytic polishing, blasting with dry ice, blasting with glass beads or other blasting material, or brushing directly in a CNC machine.
32. The method of any of clauses 27 to 31, wherein the material is stainless steel, optionally the stainless steel is an austenitic stainless steel.
33. The method of clause 32, wherein the stainless steel is one of: 1.4301; 1.4305; 1.4306; 1.4307; 1.4310; 1.4401; 1.4404; 1.4435; and 1.4441.
34. The method of any of clauses 27 to 33, wherein the material has a HRC of less than 25 HRC, or less than 20 HRC, or of between 16 and 20 HRC, or between 17 and 19 HRC, or between 17.5 and 18.5 HRC, or of about 18 HRC.

1. A method of manufacturing a cutting head for a perforator for cutting bone tissue, the method comprising the steps of:
machining a cutting head comprising a plurality of cutting edges from a material having a Rockwell C hardness HRC of less than 30 HRC; and
surface finishing the machined cutting head,
and in which the method does not comprise hardening by heat-treatment.

2. The method according to claim 1, in which the method does not comprise a step of hardening.

3. The method according to claim 1, in which the method further does not comprise a step of grinding the machined cutting head.

4. The method according to claim 1, wherein the material is a metal.

5. The method according to claim 4, wherein the material is a stainless steel, optionally wherein the material is an austenitic stainless steel.

6. (canceled)

7. The method according to claim 5, wherein the stainless steel is one of:
1.4301; 1.4305; 1.4306; 1.4307; 1.4310; 1.4401; 1.4404; 1.4435; and 1.4441.

8. The method of claim 1, comprising only the steps of: machining the cutting head from a material having a Rockwell C hardness HRC of less than 30 HRC, and surface finishing the machined cutting head.

9. The method of claim 1, wherein the material has a HRC of less than 25 HRC, optionally wherein the material has a HRC of less than 20 HRC.

10. (canceled)

11. The method according to claim 9, wherein the material has a HRC of between 16 and 20 HRC, or between 17 and 19 HRC, or between 17.5 and 18.5 HRC.

12. The method according to claim 11, wherein the material has a HRC of about 18 HRC.

13. The method claim 1, in which the step of machining the cutting head comprises one or more of: drilling, milling, single-point cutting or multi-point cutting.

14. The method according to claim 13, in which the cutting head is machined by drilling and milling, optionally computer numerical control CNC milling.

15. The method according to claim 1, wherein the cutting head and the cutting edges are machined to their final shape in a single machining step, using a single setup.

16. (canceled)

17. The method according to claim 1, wherein the step of surface finishing the machined cutting head comprises one of:
vibratory finishing; and
polishing, optionally electrolytic polishing.

18. A method of manufacturing a perforator component, comprising the steps of claim 1, and further comprising a step of providing a connector for reversibly coupling the cutting head to a drive shaft, the connector comprising a harder connector portion having a higher HRC, and a softer connector portion having a lower HRC.

19. (canceled)

20. A cutting head for a perforator for cutting bone tissue, the cutting bead comprising a plurality of cutting edges, characterised in that the cutting head consists of austenitic steel having a Rockwell C hardness HRC of less than 30 HRC.

21. The cutting head according to claim 20, wherein the austenitic steel is one of:
1.4301; 1.4305; 1.4306; 1.4307; 1.4310; 1.4401; 1.4404; 1.4435; and 1.4441.

22. The cutting head according to claim 20, wherein the austenitic steel has a HRC of less than 25 HRC, or less than 20 HRC.

23. The cutting head of claim 20, wherein the austenitic steel has a HRC of between 16 and 20 HRC, or between 17 and 19 HRC, or between 17.5 and 18.5 HRC.

24. (canceled)

25. A method of manufacturing a cutting head for a perforator, the method consisting of steps of:
machining a cutting head comprising a plurality of cutting edges from a material having a Rockwell C hardness HRC of less than 30 HRC; and
surface treating the machined cutting head.
